(19) United States
(12) Patent Application Publication
(10) Pub. No.: US 2025/0312311 A1
(43) Pub. Date: Oct. 9, 2025

(54) Low Dose Fipronil Deer Feed

(71) Applicant: Scimetrics Limited Corp., Wellington, CA (US)

(72) Inventors: Richard Poché, Wellington, CO (US); David Matthew Poché, Wellington, CO (US)

(73) Assignee: Scimetrics Limited Corp., Wellington, CO (US)

(21) Appl. No.: 18/581,087

(22) Filed: Apr. 3, 2024

(51) Int. Cl. A61K 31/415; A61K 9/00; A61K 47/46; A61P 33/14

(52) U.S. Cl. CPC A61K 31/415; A61K 9/0056; A61K 47/46; A61P 33/14

(57) ABSTRACT

A vector control composition and corresponding methods of use, the vector control composition including an active agent effective to decrease the fitness of a vector capable of transmitting a pathogen, whereby the active agent includes an acaricide or a low dose of an acaricide, whereby the active agent can be formulated for oral delivery to a host of the vector, and whereby the host can be an ungulate.

### I. BACKGROUND OF THE INVENTION

On a global scale, ticks are recognized as one of the main arthropod pathogen vectors of disease agents of humans and animals, and thus are of considerable medical importance. Ticks and wildlife species encompass vector-host relationships of increasing medical and veterinary concern, with many notable tick-borne diseases (such as anaplasmosis, babesiosis, ehrlichiosis and Lyme disease) attracting substantial medical attention. Vector control is regarded as one of the more promising means for reducing human tick bites and preventing pathogen transmission. However, conventional methods, such as area-wide broadcast applications, present management concerns, including logistical and economic hurdles, the indiscriminate targeting of non-target organisms, such as pollinators, and the accelerated development of insecticidal resistance. Accordingly, there exists a need for additional, more discriminate methods of vector control; the present invention fulfills this need and provides further related advantages.

### II. SUMMARY OF THE INVENTION

A vector control composition and corresponding methods of use, the vector control composition including an active agent effective to decrease the fitness of a vector capable of transmitting a pathogen, whereby the active agent includes an acaricide or a low dose of an acaricide, whereby the active agent can be formulated for oral delivery to a host of the vector, and whereby the host can be an ungulate.

### III. A BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A illustrates vector-host association, whereby adult females attach to white-tailed deer and blood feed for approximately 6-11 days. Fully engorged females drop off of the host and begin the reproductive process. Females then oviposit and produce thousands of eggs.

FIG. 1B illustrates the impact of fipronil deer feed (FDF) consumption by white-tailed deer on reproductive female ticks, whereby adult female ticks blood-feeding on white-tailed deer expire and are prevented from feeding to engorgement and detaching, subsequently preventing them from successfully ovipositing and reducing the reproductive rate.

FIG. 2 shows a table summarizing the test group deer utilized during the pen study and fed fipronil deer feed (FDF) or a placebo deer feed.

FIG. 3 shows fipronil deer feed (FDF) presented in an elevated deer feeder during the exposure period.

FIG. 4A is a top view of a particular embodiment of a tick capsule.

FIG. 4B is a bottom view of a particular embodiment of a tick capsule.

FIG. 5A shows female ticks being plunged into a capsule.

FIG. 5B shows a plunger being removed prior to the mesh lid being secured.

FIG. 5C shows a completed, secured capsule being checked to ensure all corners are adhered to the neck.

FIG. 5D shows a closeup of a completed capsule with 20 Ixodes scapularis mating pairs.

FIG. 6A shows deer in individual pens with completed, attached capsules.

FIG. 6B shows deer in individual pens with completed, attached capsules.

FIG. 7 shows a table with tissue classification, US Environmental Protection Agency-established maximum residue limits (MRL) and tissue identification for all tissues collected from all euthanized deer.

FIG. 8 shows a table with body weights and feed/fipronil consumption recorded for individual white-tailed deer. FDF fipronil deer feed, NA not applicable, T48 treatment group exposed to FDF for 48 h (2 days), T120 treatment group exposed to FDF for 120 h (5 days).

FIG. 9 shows a table with the total number of ticks recovered and their attachment status, feeding status and condition. These data are for both Ixodes scapularis and Amblyomma americanum introduced onto white tailed deer fed fipronil deer feed (FDF) versus untreated placebo feed. *Recovered ticks refer to ticks collected within the capsules. An additional 49 I. scapularis (12 female, 37 male), and 29 A. americanum (22 female, 7 male) were found trapped in the veterinary wrap collars. These data are excluded because it could not be determined which specific animals these ticks were parasitizing.

FIG. 10 shows the average±standard deviation (SD) weights for engorged I. scapularis females and approximate number of eggs and larvae produced within FDF treatment and control groups.

FIG. 11 shows a table with fipronil deer feed (FDF) efficacy in preventing Ixodes scapularis females from reaching engorgement and detaching.

FIGS. 12A and 12B show Ixodes scapularis and Amblyomma americanum feeding on a control deer. Although A. americanum is a larger tick than I. scapularis, the engorgement rate is markedly slower.

FIG. 13A is a table showing fipronil deer feed (FDF) efficacy in reducing Ixodes scapularis survivorship. *Includes attached and detached females alive at day 8 post-attachment.

FIG. 13B is a table showing fipronil deer feed (FDF) efficacy in reducing Amblyomma americanum survivorship. *Includes attached and detached females alive at day 8 post-attachment.

FIG. 14A shows Ixodes scapularis actively feeding on a control deer. Photo was taken at day 6 post-attachment.

FIG. 14B shows Ixodes scapularis dead and attached on a treatment deer. Photo was taken at day 6 post-attachment.

FIG. 14C shows Amblyomma americanum actively feeding on a control deer. Photo was taken at day 6 post-attachment.

FIG. 14D shows Amblyomma americanum dead and attached on a treatment deer. Photo was taken at day 6 post-attachment.

FIG. 15 shows fipronil deer feed (FDF) efficacy in reducing Ixodes scapularis and Amblyomma americanum survivorship in the treatment groups. T48, treatment group exposed to FDF for 48 h (2 days); T120, treatment group exposed to FDF for 120 h (5 days).

FIG. 16 shows a table with fipronil sulfone concentrations in plasma (Cp) for individual white-tailed deer. These data represented all deer fed FDF and untreated control deer (fed placebo), and the subsequent number of live female ticks (attached and detached I. scapularis and A. americanum) at the conclusion of the tick challenge on day 8 after tick introduction (n=40 females per deer; 20 females for each species).

FIG. 17 shows a table with Cf values for each white-tailed deer.

FIG. 18 shows a table with fipronil sulfone in white-tailed deer tissues.

FIG. 19A shows fipronil degradation in meat/muscle after consumption of fipronil deer feed (FDF). The dotted line indicates the maximum residue limits established for meat/muscle.

FIG. 19B shows fipronil degradation in meat by-products after consumption of fipronil deer feed (FDF). The dotted line indicates the maximum residue limits established for meat by-products.

FIG. 19C shows fipronil degradation in fat after consumption of fipronil deer feed (FDF). The dotted line indicates the maximum residue limits established for fat.

FIG. 19D shows fipronil degradation in liver of white-tailed deer after consumption of fipronil deer feed (FDF). The dotted line indicates the maximum residue limits established for liver.

### IV. DETAILED DESCRIPTION OF THE PREFERRED EMBODIMENTS

Disclosed herein are inventive compositions and methods of using the same to control a pathogen by controlling its vector via a vector control composition which includes a pesticide that targets the vector of the pathogen. The vector control composition can be delivered (or administered) to the vector, such as via a host of the vector, to decrease the fitness of the vector which correspondingly decreases the fitness of the pathogen to control the spread of vector-borne diseases in humans and animals.

### Pathogen Vector

The present vector control composition can target a vector comprising an acarine (an arachnid of the subclass acari) which can transmit a pathogen from one organism to another. As to particular embodiments, the vector can be a parasitic acarine. As to particular embodiments, the vector can be a tick.

Numerous tick species are significant pests which can cause harm to both humans and animals (for example, but not limited to, wildlife, livestock, farm animals, zoo animals, companion animals, or the like). Ticks are obligate, blood-feeding ectoparasites that infest the skin of another organism (referred to herein as a “host”) and depend on their host for sustenance, maturation, and proliferation. If the tick is carrying a pathogen, it can transfer this pathogen into the bloodstream of a host during blood-feeding. In addition to transmitting pathogens and their associated diseases, ticks can also elicit a local reaction at the tick bite site, whereby the host's skin may become irritated, itchy, red, and/or swollen. Further, in some cases, the tick bite site may become infected.

The present vector control composition can be employed to control any susceptible tick. As illustrative, nonlimiting examples, the vector control composition may be especially useful for controlling Ixodes scapularis (hereinafter “I. scapularis”), also known as the black-legged tick or deer tick; Amblyomma americanum (hereinafter “A. americanum”), also known as the lone star tick; Haemaphysalis longicornis (hereinafter “H. longicornis”), also known as the Asian longhorned tick; and/or Rhipicephalus microplus (hereinafter “R. microplus), also known as the cattle fever tick; however, said again, the present vector control composition is not limited to controlling only these parasitic acarines.

### Pathogen

Ticks can act as vectors for various pathogens, whereby as used herein, the term “pathogen” refers to an infectious biological agent which can cause disease and/or disease symptoms in a human or animal by, for example, directly damaging host cells, tissues, and/or organs during the process of infection; producing toxins that cause damage to host cells, tissues, and/or organs; triggering an immune/inflammatory response which may contribute to host cell, tissue, and/or organ damage; interfering with host physiological and metabolic processes; inducing hypersensitivity reactions; or the like. Pathogens transmitted by ticks can include, but are not limited to, bacteria, viruses, parasites, and protozoa.

As illustrative, nonlimiting examples, ticks can transmit Borrelia burgdorferi, a causative agent of Lyme disease; Borrelia mayonii, a causative agent of Lyme disease; Anaplasma phagocytophilum, a causative agent of anaplasmosis; Babesia microti, a causative agent of babesiosis; Ehrlichia muris eauclairensis, Ehrlichia chaffeensis, and Ehrlichiosis ewingii, causative agents of ehrlichiosis; Borrelia miyamotoi, a causative agent of Borrelia miyamotoi disease; Powassan virus, a causative agent of Powassan virus disease; Heartland virus, a causative agent of Heartland virus disease; Bourbon virus, a causative agent of Bourbon virus disease; Francisella tularensis, a causative agent of tularemia; bacteria in the genus Rickettsia, causative agents of rickettsiosis; and the causative agent (unknown) of southern tick-associated rash illness (STARI). The present vector control composition may be useful for controlling any one or more of these pathogens and/or associated diseases.

### Host

The host of the vector which the present vector control composition can target may be an ungulate, defined as a mammal characterized by the presence of hooves. There are two main groups of ungulates. The first order is Artiodactyla and includes even-toed ungulates, which are often ruminants and thus have a multi-chambered stomach for digesting plant material. Illustrative, nonlimiting examples of even-toed ungulates include deer, cattle, sheep, goats, antelope, and pigs. The second order is Perissodactyla and includes odd-toed ungulates, which are typically larger and have a more specialized digestive system compared to artiodactyls. Illustrative, nonlimiting examples of odd-toed ungulates include horses, rhinoceroses, and tapirs. As to particular embodiments, the host of the vector which the present vector control composition can target may be an African ungulate, whereby illustrative, non-limiting examples include the African elephant, the African buffalo, the giraffe, the wildebeest, the zebra, the antelope, the rhinoceros, and the hippopotamus.

As an ungulate example, deer (family Cervidae) can serve as a potential blood-meal host for several medically important tick species, including I. scapularis, A. americanum, H. longicornis, and R. microplus. By hosting ticks, deer can contribute to the maintenance and spread of tick populations, thus influencing the risk of tick-borne disease transmission in their habitats.

An exponential increase in white-tailed deer (Odocoileus virginianus) populations and geographical distribution has been linked to an increase in I. scapularis abundance and geographical distribution and the subsequent rise in the incidence of Lyme disease. These coinciding increases can be attributed to white-tailed deer acting as the primary breeding sites of I. scapularis, with approximately 90% of adult I. scapularis being estimated to feed on deer. Accordingly, white-tailed deer represent a key reproductive host for this tick species.

The increase in deer populations has also been linked with an increase in A. americanum populations, another medically important tick species which parasitizes white-tailed deer at various stages of the tick life cycle (adults, nymphs, larvae) and is heavily reliant on this host for reproduction and development.

Attempts have been made to control parasitizing ticks by targeting white-tailed deer with topical pesticides using the federally approved the 4-Poster Deer Feeder. This device is comprised of a centralized bin containing whole kernel corn utilized for bait, with attached feeding stations and appendages on each side of the central bin. The appendages hold paint roller applicators made from fibrous material, which rotate on PVC pipes connected to the adjacent feeding stations. A plate partially occludes each feeding station, forcing contact between the pesticide-charged applicators and the deer as they feed. Subsequently, as deer feed, the paint roller applicators transfer the pesticide onto the head, neck, and ears of the deer, which disperses to the remainder of the body through self-grooming. However, a number of issues have limited the use of this technology, including the labor and maintenance required to service the device (refilling corn, applying pesticide to rollers, fixing broken rollers, etc.). Further, conducted studies have shown a lack of success with this device in impacting overall instances of Lyme disease.

As a more direct, practical, and less cumbersome approach relative to topical administration, the present invention generally comprises orally delivering a pesticide to a host via a feed for the host which includes the pesticide to consequently deliver the pesticide to a vector which blood-feeds on the host, whereby the pesticide can be effective to decrease the fitness of the vector. As to particular embodiments, the present invention comprises orally delivering an active agent to an ungulate via a feed for the ungulate which includes the active agent to consequently deliver the active agent to a tick which blood-feeds on the ungulate, whereby the active agent can be effective to decrease the fitness of the tick. As to particular embodiments, the present invention comprises orally delivering an active agent to a deer via a feed for the deer which includes the active agent to consequently deliver the active agent to a tick which blood-feeds on the deer, whereby the active agent can be effective to decrease the fitness of the tick.

For such oral delivery to a host, such as an ungulate or deer, the present vector control composition can be formulated as a feed which contains an active agent effective to decrease the fitness of a tick capable of transmitting a pathogen.

### Vector Control Composition

As used herein, the term “vector control composition” refers to a composition including an active agent effective to control or decrease the fitness of a vector capable of transmitting a pathogen.

As used herein, the term “active agent” refers to a component of a composition which is responsible for and/or contributes significantly to the intended physiological effect or outcome following delivery of the composition.

As used herein “decreasing the fitness of a vector” refers to any disruption in the physiology of a vector and/or activity of a vector as a consequence of delivery of the vector control composition described herein, including but not limited to any one or more of the following desired effects: (1) killing a vector or decreasing the lifespan of a vector by greater than about 10%, 20%, 30%, 40%, 50%, 60%, 70%, 80%, 90%, 95%, 99%, or 100%; (2) decreasing a vector population by greater than about 10%, 20%, 30%, 40%, 50%, 60%, 70%, 80%, 90%, 95%, 99%, or 100%; (3) decreasing the growth or body weight or body mass of a vector by greater than about 10%, 20%, 30%, 40%, 50%, 60%, 70%, 80%, 90%, 95%, 99%, or 100%; (4) decreasing the development of a vector by greater than about 10%, 20%, 30%, 40%, 50%, 60%, 70%, 80%, 90%, 95%, 99%, or 100%; (5) decreasing the competence of a vector by greater than about 10%, 20%, 30%, 40%, 50%, 60%, 70%, 80%, 90%, 95%, 99%, or 100%; (6) decreasing the metabolic rate or activity of a vector by greater than about 10%, 20%, 30%, 40%, 50%, 60%, 70%, 80%, 90%, 95%, 99%, or 100%; (7) decreasing the mobility of a vector by greater than about 10%, 20%, 30%, 40%, 50%, 60%, 70%, 80%, 90%, 95%, 99%, or 100%; (8) decreasing the reproductive rate of a vector by greater than about 10%, 20%, 30%, 40%, 50%, 60%, 70%, 80%, 90%, 95%, 99%, or 100%; (9) decreasing pathogen transmission (vertical or horizontal transmission) by a vector by greater than about 10%, 20%, 30%, 40%, 50%, 60%, 70%, 80%, 90%, 95%, 99%, or 100%; and/or (10) increasing susceptibility of a vector to a pesticide(s) by greater than about 10%, 20%, 30%, 40%, 50%, 60%, 70%, 80%, 90%, 95%, 99%, or 100% or more. A decrease in vector fitness can be determined in comparison to an untreated vector.

As used herein, the term “untreated vector” refers to a vector that has not been exposed to, contacted with, and/or delivered the vector control composition, including (i) a separate vector that has not been exposed to, contacted with, and/or delivered the vector control composition, or (ii) the same vector undergoing treatment assessed at a time point prior to exposure to, contact with, and/or delivery of the vector control composition.

Notably, decreasing the fitness of a pathogen to control the spread of the pathogen can be a consequence of delivery of the vector control composition to a vector carrying the pathogen, as a decrease in the fitness of the vector correspondingly decreases the fitness of the pathogen carried by the vector, thereby interrupting the transmission cycle of the pathogen which can reduce the incidence and prevalence of vector-borne diseases. As illustrative examples, decreasing the fitness of a pathogen may manifest as a deterioration or decline in the physiology of the pathogen as a consequence of delivery of the vector control composition to the vector carrying the pathogen. In some instances, the fitness of a pathogen may be measured by one or more parameters, including but not limited to survival, viability, lifespan, health, body weight, metabolic rate or activity, development, mobility, fertility, or reproductive rate in comparison to an untreated pathogen. In some instances, the decrease in pathogen fitness may manifest as an increase in the pathogen's sensitivity to an antipathogen agent and/or a decrease in the pathogen's resistance to an antipathogen agent in comparison to an untreated pathogen. Further, in some instances, the decrease in pathogen fitness may manifest as other fitness disadvantages, such as a decreased tolerance to certain environmental factors (for example, a high or low temperature tolerance), a decreased ability to survive in certain habitats, or a decreased ability to sustain a certain diet in comparison to an untreated pathogen.

The vector control composition of the present invention includes an acaricide which can be effective to decrease the fitness of an acarine vector. As to particular embodiments, the acaricide can be a pyrazole acaricide. As to particular embodiments, the pyrazole acaricide can be a phenylpyrazole acaricide. As to particular embodiments, the phenylpyrazole acaricide can be acetoprole. As to particular embodiments, the phenylpyrazole acaricide can be vaniliprole. As to particular embodiments, the phenylpyrazole acaricide can be fipronil (CAS Number: 120068-37-3), which has a molecular formula of C₁₂H₄C₁₂F₆N₄OS and the structure shown in Formula I.

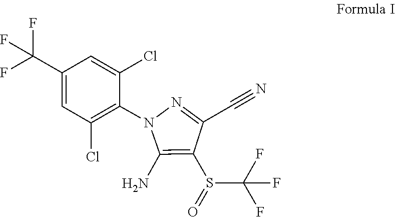

Fipronil is a broad-spectrum acaricide which targets the gamma-aminobutyric acid (GABA) receptor and glutamate-gated chloride channels in the central nervous system of acarines, both of which play a crucial role in regulating nerve impulses. When fipronil binds to these receptors, it blocks the passage of chloride ions through the channels, leading to hyperexcitation of the nervous system, ultimately causing paralysis and death of the acarine.

Mammals, including humans, also have GABA receptors and glutamate-gated chloride channels, but the structure of these receptors differs between acarines and mammals. Fipronil has a higher affinity for acarine receptors compared to mammalian receptors, which means fipronil is more selective in its action on acarine nervous systems and less likely to affect mammals. This selective action on acarine receptors makes fipronil an effective acaricide while minimizing its impact on non-target organisms, including mammals.

As to particular embodiments, the vector control composition of the present invention can include fipronil in an amount effective to decrease the fitness of an acarine vector, whereby this amount can be a relatively low dose of fipronil.

It will be appreciated that minimizing the amount of active agent (such as fipronil) in the vector control composition is advantageous for many reasons, for example (i) because it decreases the amount of active agent introduced into the environment; (ii) because it reduces the risk of non-target organisms ingesting a lethal dose of the active agent; (iii) because it may decrease the overall cost of the vector control composition; and (iv) because it may decrease the risk associated with preparing and handling the vector control composition.

As to particular embodiments, the vector control composition can include a relatively low dose of fipronil which can be effective to decrease the fitness of an acarine vector upon ingestion of the low-dose fipronil by the acarine vector. Correspondingly, the low-dose fipronil can be formulated for oral delivery to the acarine vector. As to particular embodiments, for oral delivery to the acarine vector, the low-dose fipronil can be formulated for ingestion by the acarine vector during blood-feeding on a host.

Additionally, the relatively low dose of fipronil can be effective to decrease the fitness of an acarine vector upon ingestion of the vector control composition by a host. Correspondingly, the vector control composition can be formulated for oral delivery to a host. After a host ingests the vector control composition, at least a portion of the fipronil can be absorbed into the host's bloodstream and thus, can be systemically distributed. Consequently, when blood-feeding on the host, the acarine vector can incidentally ingest fipronil present in the blood of the host.

As to particular embodiments, the vector control composition can include an amount of fipronil selected from the group including or consisting of: not greater than about 0.005% by weight of the composition; less than about 0.005% by weight of the composition; not greater than about 0.0049% by weight of the composition; not greater than about 0.0045% by weight of the composition; not greater than about 0.004% by weight of the composition; not greater than about 0.0035% by weight of the composition; not greater than about 0.003% by weight of the composition; not greater than about 0.0025% by weight of the composition; not greater than about 0.002% by weight of the composition; not greater than about 0.0015% by weight of the composition; and not greater than about 0.001% by weight of the composition. Upon ingestion, this amount of fipronil can be effective to decrease the fitness of an acarine vector.

As to particular embodiments, the vector control composition can include an amount of fipronil selected from the group including or consisting of: a range of between about 0.001% and about 0.005% by weight of the composition; a range of between about 0.001% and less than about 0.005% by weight of the composition; a range of between about 0.001% and about 0.0049% by weight of the composition; a range of between about 0.001% and about 0.0045% by weight of the composition; a range of between about 0.001% and about 0.004% by weight of the composition; a range of between about 0.0015% and about 0.0035% by weight of the composition; and a range of between about 0.002% and about 0.003% by weight of the composition. Upon ingestion, this amount of fipronil can be effective to decrease the fitness of an acarine vector.

As but one illustrative example, the vector control composition can include about 0.0025% fipronil by weight of the composition. Upon ingestion, this amount of fipronil can be effective to decrease the fitness of an acarine vector.

In addition to effectively decreasing the fitness of a vector, as to particular embodiments, the present vector control composition including a relatively low dose of fipronil can further be effective to prevent an infection (the presence or colonization of a pathogen in, on, or around an organism) in an organism at risk of such an infection with a pathogen, particularly when the infection can decrease the fitness of the organism, e.g., by causing disease, disease symptoms, or an immune/inflammatory response. As used herein, the term “prevent an infection” refers to preventing the onset of an infection and/or symptoms or conditions associated with an infection.

### Delivery

A vector can be exposed to the active agent of the vector control composition described herein in any suitable manner that permits delivering the active agent to the vector. Following, the method of decreasing the fitness of a vector capable of transmitting a pathogen includes delivering the active agent to the vector.

As stated above, as to particular embodiments, the vector control composition can be formulated for oral delivery to a host of the vector. To reiterate, after the host ingests the vector control composition, at least a portion of the active agent can be absorbed into the host's bloodstream and thus, can be systemically distributed; when blood-feeding on the host, the vector can incidentally ingest the active agent present in the blood of the host.

As to particular embodiments, for oral delivery to a host, the vector control composition can be formulated as a comestible composition for ingestion by the host. As to particular embodiments, the vector control composition can be formulated as comestible matter, such as feed (or food or diet), which can be ingested by a host; such a formulation can be relatively easy to deliver and may increase uptake of the vector control composition by the host. In addition, such a formulation can be particularly suited for chronic dosing resulting from multiple feedings, which may be in contrast to acute dosing.

As used herein, the term “feed” refers to the combination of at least a purposely selected amount of an active agent and a purposely selected carrier for use as a targeted vector control composition, which is to be contrasted with, for instance, a naturally occurring material. The feed should be both palatable to a target host and edible by the target host.

As to particular embodiments, the feed can comprise at least an amount of a low dose of an acaricide and an acceptable carrier (or excipient), such as a pharmaceutically acceptable carrier suitable for administration to an animal and of course, nontoxic to the animal in the amount employed. The carrier should be palatable to a target host and thus, can comprise any of a numerous and wide variety of edibles, a list of which is herein contemplated but too extensive to include.

As but one illustrative, non-limiting example, the carrier can comprise sugar beets or a sugar beet formulation. Sugar beets are primarily valued for their high sugar content (predominantly sucrose), which typically ranges from 15% to 20% of their total weight. Also, sugar beets can contain fiber, various vitamins and minerals (such as vitamin C, folate, potassium, magnesium, and iron), nitrate, betaine, phytonutrients (such as betalains which are responsible for their characteristic red color), and protein.

As but a second illustrative, non-limiting example, the carrier can comprise cornmeal or a cornmeal formulation. Cornmeal, made from ground corn kernels, is primarily composed of carbohydrates, mainly in the form of starch. Additionally, cornmeal can contain protein, fiber, vitamins (such as B vitamins including thiamine (vitamin B1), niacin (vitamin B3), and folate (vitamin B9), minerals (such as magnesium, phosphorus, and potassium), fat (primarily in the form of unsaturated fats), and antioxidants (such as carotenoids).

As to particular embodiments, the feed can also include one or more attractants, such as a flavorant, a palatant, an odorant, or the like, for attracting a host. The attractant may be embodied by the carrier or can be discrete from the carrier, depending upon the formulation. As illustrative, non-limiting examples, for a herbivorous target host, the attractant can be a plant-derived component, such as corn, corn products, corn meal, wheat, wheat products, oats, oat products, cereals, sugars, syrups, fruits, vegetables, seeds, nuts, or the like.

As to particular embodiments, the carrier, the attractant, or both, can be selected with consideration for non-target organisms to reduce potential uptake of the feed by the non-target organisms. Accordingly, the selection of the particular type and amount of carrier, attractant, or both, may vary depending upon the non-target organisms desired to deter. Furthermore, as to particular embodiments, the feed can also include one or more repellants intended to repel the non-target organisms.

As to particular embodiments, the feed can, but need not necessarily, further include a colorant or dye which can visibly color the feed, thereby allowing visible identification of feed including the colorant.

As to particular embodiments, the colorant can be taken up by tissue of a host following ingestion to visibly color the tissue, thereby providing a visible indicium to indicate that the host has ingested the feed, which may discourage consumption of the host.

The feed can, but need not necessarily, include more than one carrier and attractant, and can further include additional components, such as additives, preservatives, plasticizers, humectants, buffering agents, or the like.

As to particular embodiments, the vector control composition formulated as feed can be in a substantially solid form, meaning firm and stable in shape (not fluid or liquid). As illustrative, nonlimiting examples, the feed can be in the form of a powder, granules, pellets, briquettes, bricks, blocks, or the like. It will be appreciated that the actual shape of the feed may not be a crucial parameter, and that any obtainable shape is within the scope of the present invention.

As to particular embodiments, the vector control composition formulated as feed can further be substantially homogenous or homogenous, meaning of uniform composition throughout.

The vector control composition formulated as feed may be an immediate-release formulation, an extended release formulation, or a delayed-release formulation, depending upon the embodiment.

To provide the vector control composition formulated as feed, the active agent can be combined with a carrier. For example, an amount of a low dose of an acaricide can be incorporated into the carrier or dispersed throughout the carrier, which may be accomplished by combining or mixing the desired amount of a low dose of an acaricide and the desired amount of a carrier.

Amounts and locations for disposition of the vector control composition described herein can be generally determined at least by the habits of the vector and/or the host as well as the physical and functional characteristics of the vector control composition.

### EXAMPLE

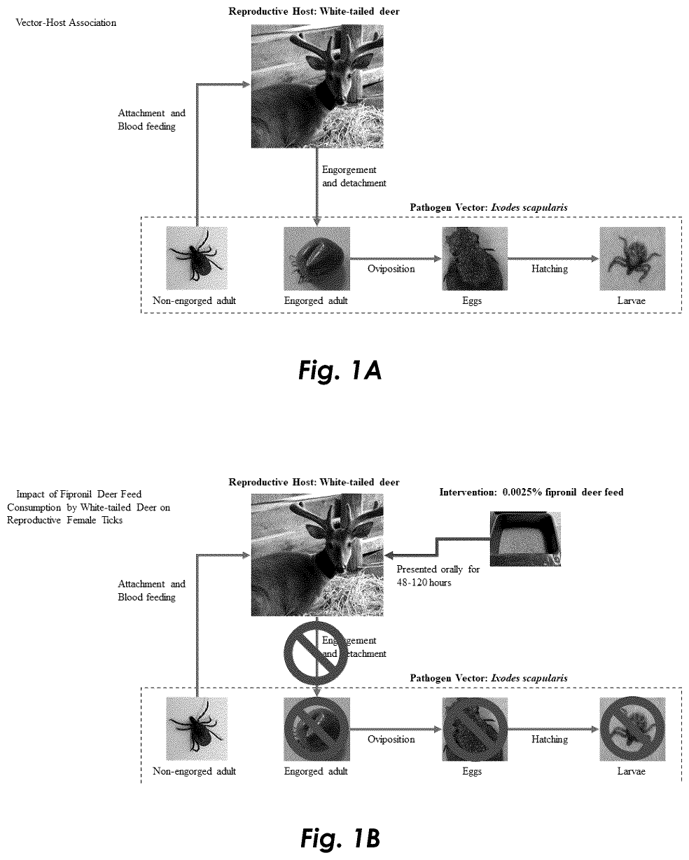

The primary objective of the study described herein was to evaluate the efficacy of an inventive vector control composition, hereafter referred to as “fipronil deer feed,” against I. scapularis and A. americanum ticks parasitizing white-tailed deer under pen conditions. The vector-host association and treatment concept are presented in FIG. 1. I. scapularis was selected because it is a vector of seven human pathogens, with the most notable being those causing Lyme disease. Lyme disease is the most common vector-borne disease in the USA, occurring most frequently in the Northeast and Midwest of the USA, and is estimated to account for approximately 500,000 human cases per year. A. americanum was selected because it is suspected to vector five or more disease agents transmissible to humans, and is also linked with STARI and red meat allergy.

### Fipronil Deer Feed

The inventive fipronil deer feed or FDF comprises a granular sugar beet formulation that was considerably palatable to white-tailed deer with a nominal fipronil concentration of 0.0025%, which was determined to control 100% I. scapularis parasitizing white-tailed deer at 24 h post-exposure when they were presented with FDF for 48 h. To produce FDF, the raw ingredients were mixed in an industry-standard electronic mixer.

### Pre-Fipronil Deer Feed Exposure (Acclimation)

Both male and female adult and yearling test deer were utilized. At the initiation of acclimation, test deer were transferred from group paddocks into the handling barn where they were maintained individually in approximately 7.5 m (length)×3 m (width) pens. The roofs of the pens were partially opened allowing for sunlight, but also had canopies to shield the test deer and FDF from inclement weather. The walls of the pens were high enough to prevent deer from escaping (approx. 3 m). Deer were acclimated to test conditions for 3 days prior to FDF exposure, and the general health of all deer was monitored daily. During this time, deer were presented with commercial deer diet (PSU Breeder 18% Deer Diet; Cargil Animal Nutrition, Minneapolis, MN, USA) ad libitum and each day were presented with approximately 500 g of untreated feed containing all of the inactive ingredients in the FDF (placebo). All deer were examined by a veterinarian prior to FDF exposure.

### White-Tailed Deer Test Group and Subgroup Assignment

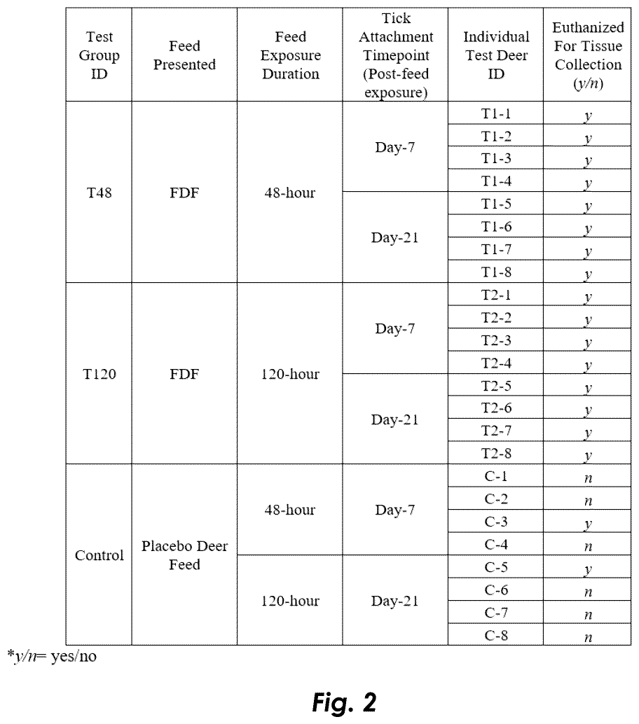

Deer were assigned to groups using a random sequence generator, and groups were differentiated based on: (i) test group identity (treatment group [T], control [C]); and (ii) the length of the exposure (48 h [T48], 120 h [T120]) (FIG. 2). While explicit guidelines for white-tailed deer are not available, federal guidelines recommend a sample size of 6-10 subjects per test group when evaluating pesticides against pests of humans and pets, such as fleas and ticks. The size of the captive herd and the number of deer that its managers could afford to donate to this project limited the sample size, and it was not possible to have an equal number of males (n=15) and females (n=9). It was determined that each test group could comprise eight animals (n=24). A total of 16 deer were offered FDF, with eight deer being exposed to FDF for 48 h and eight deer being exposed to FDF for 120 h. An additional eight deer served as an untreated control group, with 50% of animals exposed to placebo for 48 h and 50% exposed to placebo for 120 h. Deer continued to be housed in the individual pens during the exposure period. Prior to tick attachment, deer within each test group were additionally assigned to subgroups, with 50% of deer to be parasitized with ticks at day 7 post-exposure to FDF, and 50% to be parasitized at day-21 post-exposure to FDF (4 animals/subgroup).

### FDF Exposure

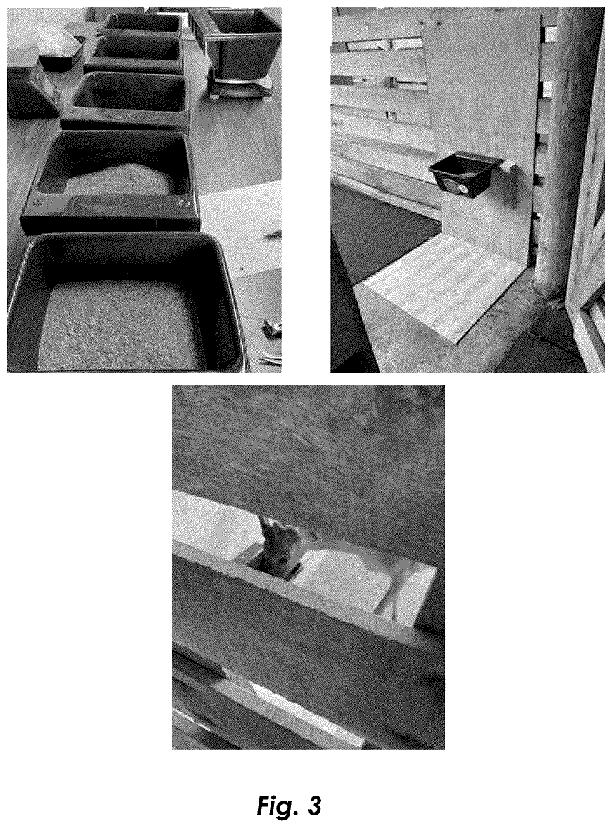

At the initiation of the deer feed exposure period, deer were presented exclusively with FDF in an elevated livestock feeder (FIG. 3). A maximum of 1 kg was presented to deer every 24 h, and deer were provided commercial deer diet immediately upon consuming all FDF. Each morning of exposure (08:00 a.m.) the FDF was removed temporarily and weighed to the nearest 0.1 g, after which fresh FDF was immediately presented to the deer. At the conclusion of the exposure period, all FDF was weighed and permanently removed. The above procedures were also followed for the deer in the control group, but they were presented with an untreated placebo (containing all ingredients of FDF minus fipronil) rather than FDF.

### Post-Deer Feed Exposure Deer Handling

At the conclusion of exposure, all FDF or placebo was removed, and deer were released into group paddocks until tick attachment. Deer were fed on a commercial diet exclusively ad libitum for the remainder of the study. During the post-exposure period, but prior to tick attachment, deer remained in the group paddocks and their general health was observed daily.

### Tick Attachment

Regarding ticks, equal numbers of each sex and species (I. scapularis and A. americanum) were obtained. Using standardized PCR assays, I. scapularis were screened for B. burgdorferi and Anaplasma phagocytophilum, and A. americanum were screened for the presence of Ehrlichia chaffeensis, Francisella tularensis, and Rickettsia rickettsii. All PCR-screened ticks were negative for the above pathogens. The ticks were housed in an industry-standard desiccator with the relative humidity maintained at >90% until enclosed in a feeding capsule for attachment to deer.

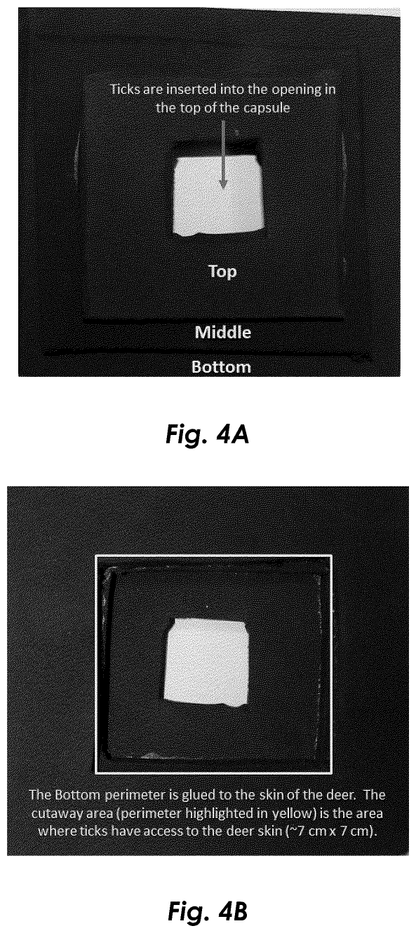

Inventive feeding capsules specifically designed for holding blood-feeding I. scapularis and A. americanum were utilized in this study. Feeding capsules allow for the containment and localization of ticks and aid in facilitating blood feeding. To make each capsule, sheets of ethylene-vinyl acetate foam were cut into three square pieces. Each square had a different outside area, allowing for flexibility (base, approx. 12×12 cm; middle, approx. 9×9 cm; top, approx. 7×7 cm), and had a combined depth of approximately 18 mm. The center of each square was cut away, creating an opening. The inner surface areas of the base and middle piece openings were each approximately 7×7 cm; the top piece had a smaller opening (approx. 1.5×1.5 cm) through which the ticks were to be inserted, which decreased the probability that ticks would escape through the top of the capsule (FIGS. 4A and 4B).

Deer were anesthetized using an intramuscular injection of telazol and xylazine at dosages of approximately 3 mg/kg and approximately 2.5 mg/kg, respectively. Once fully anesthetized, deer were weighed to the nearest 0.1 kg using a certified balance. Prior to blood collection and capsule attachment, large patches of fur on the neck were trimmed using electric horse clippers. Prior to capsule attachment, 10 ml of blood was collected from the jugular vein of each deer using a 20-gauge needle. The blood from each individual deer was immediately placed into a vacutainer containing EDTA and was centrifuged for 10 min at 7000 revolutions/min. The plasma was transferred to 1.5-ml centrifuge tubes, which were then stored at −20° C. until analysis.

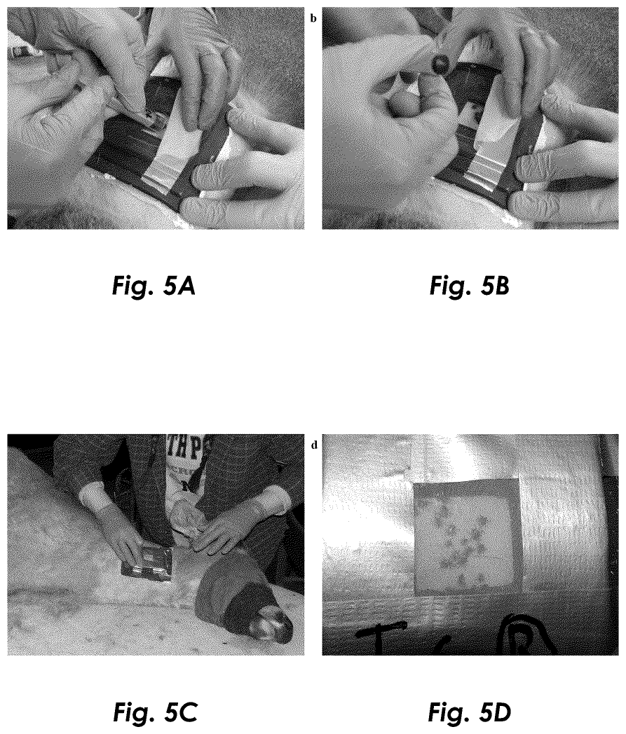

Two identical tick feeding capsules were attached to opposing sides of the neck of each deer using a liberal amount of fabric glue. Each capsule was held firmly in place for >3 min to allow it to adhere to the skin and fur. For each deer, 20 I. scapularis mating pairs were placed within one capsule, and 20 A. americanum mating pairs were placed within the second capsule. Prior to tick attachment, 20 ticks (all same species and sex) were placed into a modified 5 ml syringe. Ticks were chilled in ice for approximately 5-10 min to slow movement. The 20 mating pairs were then carefully plunged into the capsules and a fine mesh lid was applied and reinforced with duct tape. Representative photos of the tick attachment process are presented in FIG. 5. The capsules were further secured to deer by wrapping the neck with a veterinary bandage.

After completion of capsule and tick attachment, deer were given tolazine via intramuscular injection at a dose of 4 mg/kg to reverse the effects of the anesthetic. Deer were then housed in individual pens, observed closely until they were mobile and moving normally and monitored routinely for the remainder of the day.

### Post-Attachment

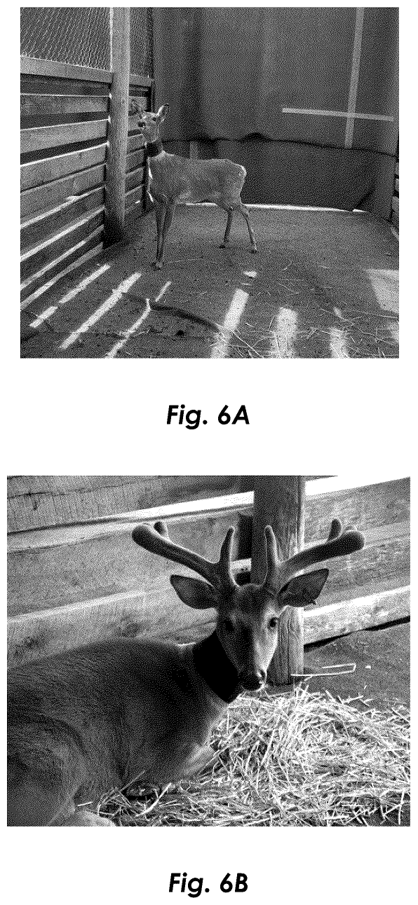

The post-attachment period spanned the initial 8 days following tick attachment (day 0 to day 8). During this time, deer were housed individually in pens (FIGS. 6A and 6B) and checked daily to ensure adequate health and wellbeing and to ensure that the capsules remained firmly attached. At day 6 and day 8 post-attachment, the deer were anesthetized in the previously described manner, and the capsules were opened to monitor the condition of the ticks. These time points were selected because I. scapularis was the primary species of concern and reportedly takes approximately 6-11 days to reach engorgement and detach.

Ticks were easily visible by the naked eye. The inside of each capsule was carefully scanned for attached and detached ticks. The numbers of total ticks recovered and their attachment status (attached, detached), feeding status (flat, partially engorged, fully engorged) and condition (alive, dead) were recorded. Any dead ticks (attached, detached) were removed from deer. At the conclusion of tick observations on day 8 post-attachment, capsules were completely removed, and any live or dead ticks were manually removed from the deer.

Fully engorged, live detached female ticks were collected, weighed to the nearest 0.0001 g using an analytical balance, and maintained individually in vials. Engorged females were maintained in a desiccator (>90% relative humidity) and were allowed approximately 14-28 days to complete oviposition. After oviposition was completed, females were removed, and egg masses weighed to the nearest 0.0001 g. Egg masses were monitored for the emergence of larvae, with eggs embryonating within approximately 35-50 days. Egg masses were monitored for approximately 2-3 weeks to estimate the proportion of hatched eggs.

### Deer Tissue Collection

At the conclusion of the tick observations on day 8 post-attachment, fresh fecal samples were collected from each test deer pen. Additionally, internal tissues were collected from each deer in each treatment group. The deer were first sedated by injection of 1-2 mg/kg xylazine hydrochloride (100 mg/ml) into the large muscle bellies of the rump/rear limbs. While sedated, deer were euthanized by intravenous injection, administered via the jugular vein, of 86 mg/kg Euthasol (pentobarbital sodium, 390 mg/ml), resulting in pentobarbital sodium overdose. Death was confirmed by a combination of the following: (i) lack of heartbeat based on auscultation with a stethoscope; (ii) lack of respiration based on visual inspection of the thorax; (iii) lack of corneal reflex; and (iv) lack of response to firm toe pinch.

Various tissues were collected from euthanized deer. The objective was to collect tissues similar to what would be collected by hunters when field dressing a killed deer. Thus, the focus was on specific meat cuts, meat by-products and fatty tissues. Approximately 50 g of each tissue was surgically removed using disposable scalpels. Scalpels and surgical gloves were replaced between each individual tissue collection to minimize the risk of contamination. Each tissue was transferred to an individual biological specimen bag, which was immediately stored at −20° C. until analysis. In addition to collecting tissues from 16 deer in the treatment group, tissues were collected from two deer in the control group to establish a baseline and for analytical method development.

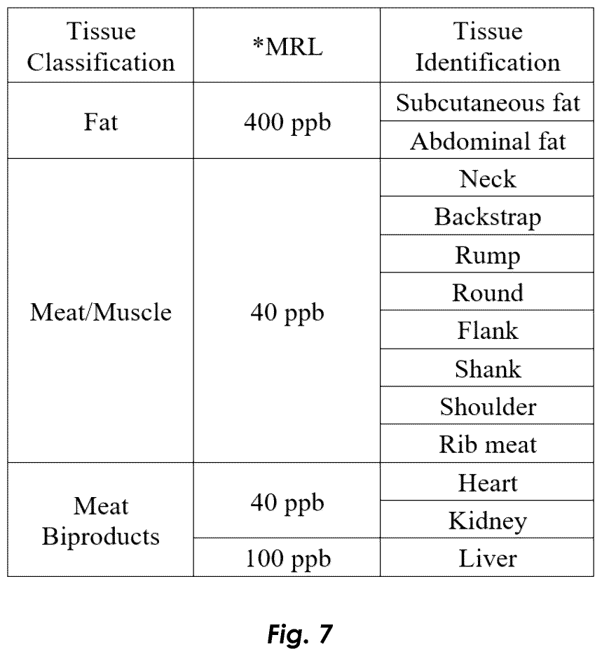

Tissues, plasma and feces were analyzed for the presence of fipronil and fipronil metabolites using validated methods of liquid chromatography/mass spectrometry (LC/MS). A list of tissue classifications, the maximum residue limits (MRL) listed by the US Environmental Protection Agency (EPA) for fipronil in cattle and the explicit tissue identifications are presented in FIG. 7.

### FDF Consumption and Deer Body Weights

The amount of FDF consumed by each deer was calculated daily and recorded to the nearest 0.1 g. The total fipronil consumed by each deer (mg) and body weights (kg) recorded prior to tick attachment were used to estimate the amount of fipronil (in mg) consumed per kilogram deer. Differences in daily FDF/placebo consumption and deer body weights between groups were compared using an analysis of variance. Differences in total fipronil consumed per deer (mg/kg) were compared between T48 and T120 using a Student's t-test.

### Tick Observations and Recovery

Adult ticks observed and counted at day 6 and day 8 post-exposure were defined by attachment status (attached, detached) and feeding status (non-engorged, partially engorged, fully engorged), with ‘attached’=adults which remain imbedded in the skin of deer; ‘detached’=adults which are not imbedded in the skin of deer; ‘flat’=non-engorged adults, showing no discernable blood meal; ‘partially engorged’=adults with partial blood meal discernable, but not fully fed; and ‘fully engorged’=completely bloated and darkly colored adults. Ticks were further defined by condition (dead, alive), which was determined by carefully observing and manipulating attached and detached ticks with fine-tipped forceps to elicit movement, with ‘alive’=movement of legs, palps or mouthparts; and ‘dead’=no movement after approximately 45 s of manipulation. The attachment status, feeding status and condition of female ticks were compared between treatment and control groups. The proportion of ticks recovered within each test group was also investigated. Differences in the proportion of ticks attached and detached for each species and differences in feeding status and condition of each species within each test group were compared using a Pearson's x² test for independence.

The weights of engorged females and approximate number of eggs and hatched larvae were compared between the treatment and control groups. To estimate the approximate number of eggs in each egg mass, an assumption was made that 1 g of ixodid eggs would contain approximately 20,000 individual eggs. The number of hatched larvae was estimated by multiplying the approximate proportion of hatched eggs by the approximate number of eggs. Differences in the weights of engorged females detaching from FDF-treated deer, relative to deer in the control group, and the subsequent numbers of eggs and larvae produced per female were estimated using a Student's t-test.

### Mortality Estimates

Mortality/efficacy in controlling I. scapularis and A. americanum was evaluated post-attachment. Two metrics were used to evaluate efficacy: (i) the average number of live engorged females successfully detaching by day 8 post-attachment; and (ii) the average survivorship of females (attached and detached) at the conclusion of day 8 post-attachment.

Efficacy of FDF in controlling blood-feeding I. scapularis and A. americanum, relative to the untreated control groups, was estimated using Abbott's formula:

$$Efficacy ( \% ) = 100 * ( \frac{C - T}{C} )$$

where T=n treatment group, and C=n control group.

### Fipronil Concentration in Deer Samples

The concentrations of fipronil and fipronil metabolites in plasma (Cp) (LOQ=0.04 ppb) and feces (Cf) (LOQ=0.1 ppb) were estimated for each individual deer (n=24). Linear regression (P<0.05) was used to detect a correlation between Cp or Cf (dependent) and the mg fipronil/kg body weight consumed by white-tailed deer (independent). Linear regression was also used to detect a potential correlation between Cp and survivorship of female I. scapularis and A. americanum ticks.

The concentrations of fipronil and fipronil metabolites within various tissues (Ct) were estimated for 16 FDF-treated deer and two control deer (LOQ=0.04 ppb). Differences in Ct values among all tissue classifications (fat, meat, meat by-products, liver) were estimated using a Kruskal-Wallis H-test followed by a Wilcoxon signed-rank test within each pair. Differences in Ct values between the T48 and T120 exposure groups estimated for each tissue classification and differences in Ct values of each tissue classification within each test subgroup were estimated using a Wilcoxon signed-rank test. The Ct was compared with the MRL established by the US EPA for ruminant cattle [Abbott W S. A method of computing the effectiveness of an insecticide. J Econ Entomol. 1925; 18:265-7] (meat/muscle=40 ppb; liver=100 ppb; meat biproducts=40 ppb; fat=400 ppb) which are utilized by the US Food and Drug Administration (FDA) when evaluating potential products. The Ct values recorded at each time point post-exposure (day 15, day 29) were used to develop exponential equations to approximate the rate of fipronil degradation for each tissue classification as a function of the number of days post-exposure. The equation was formulated as follows, and is functionally similar to equations previously utilized by Poché et al. [Poché D M, Grant W E, Wang H. Visceral leishmaniasis on the Indian subcontinent: modelling the dynamic relationship between vector control schemes and vector lifecycles. PLoS Negl Trop Dis. 2016; e0004868] to represent fipronil degradation in bovid plasma and feces:

$$Fipronil Degradation = {θ}^{1} * EXP ( {θ}^{2} x )$$

where θ¹=Theta-1 estimate, θ²=Theta-2 estimate, EXP=exponential, x=days post-exposure.

All analyses were performed using the current versions of JMP statistical software (version 15) and Microsoft Excel. Differences were considered significant if P<0.05.

### FDF Consumption and White-Tailed Deer Body Weights

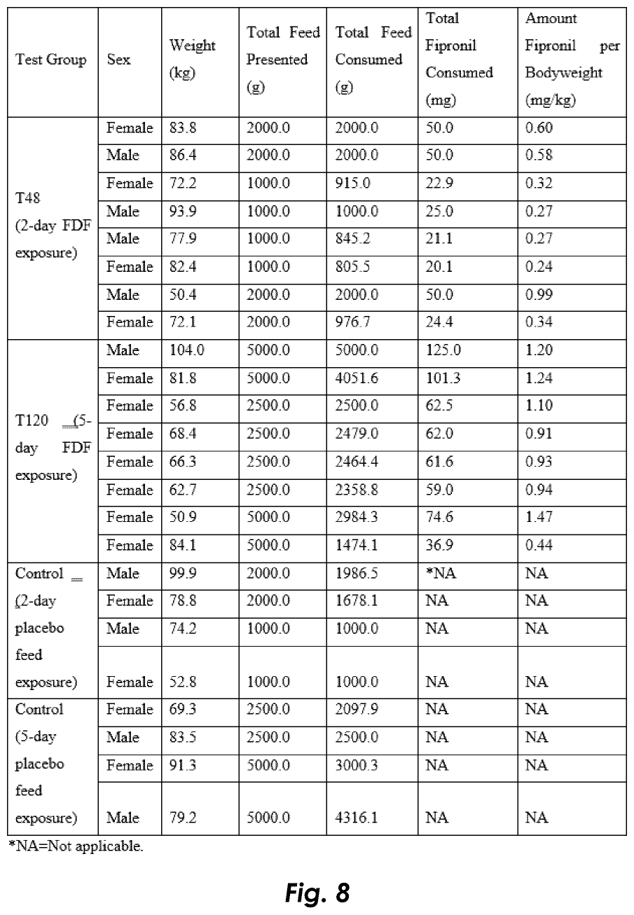

A total of 24-deer were utilized in this study, and all deer appeared to be healthy throughout the experiment. Individual deer body weights, total FDF consumption and fipronil consumed are presented in FIG. 8. For the 48-h exposure group, FDF consumption (g), body weight (kg) and fipronil consumption (mg/kg) ranged from 805.5 to 2000 g, from 50.4 to 93.9 kg and from 0.24 to 0.99 mg/kg, respectively. For the 120-h exposure group, FDF consumption (g), body weight (kg) and fipronil consumption (mg/kg) ranged from 1474.1 to 5000.0 g, from 50.9 to 104 kg and from 0.44 to 1.47 mg/kg, respectively.

No significant differences were found when comparing daily FDF and placebo consumption at 48-h exposure and 120-h exposure. No significant differences were detected when comparing individual deer body weights among test groups. As expected, the amount of fipronil consumed (mg/kg) by each deer was significantly higher in T120 (5-day exposure) than in T48 (2-day exposure) (t_{(13.636)}=4.082, P=0.0012).

### Tick Recovery and Attachment Status

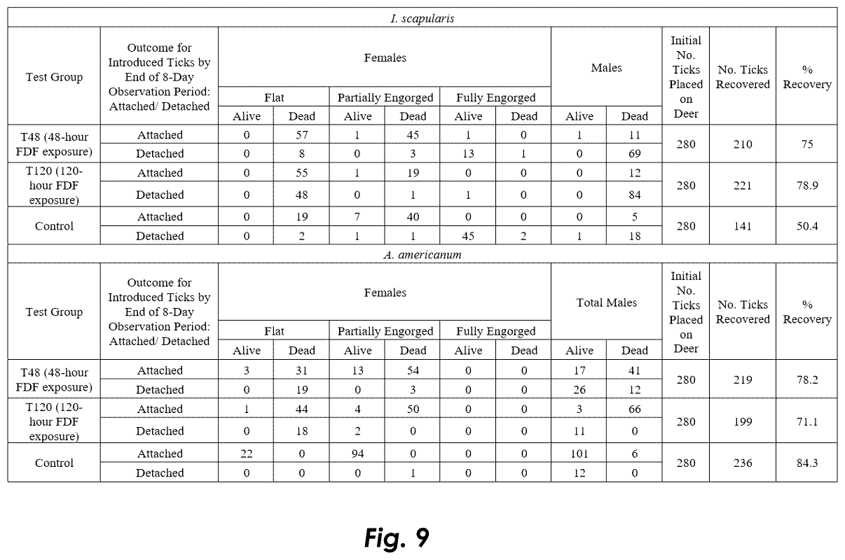

The system of containing and recovering adult ticks infesting deer was relatively efficient. Tick recovery and attachment status data are explicitly represented in FIG. 9 and were used to calculate all sums and percentages presented in this section. In total, white-tailed deer were infested with 840 I. scapularis (420 female, 420 male) and 840 A. americanum (420 female, 420 male) ticks. Of these 1680 ticks, 1226 were recovered during the post-attachment period (73%).

The probability of I. scapularis females detaching or remaining attached was significantly different relative to A. americanum (x²=42.243, P<0.0001), with I. scapularis being more likely to detach over the 8-day observation period. Males of both tick species showed a significantly greater tendency to be detached than did females (x²=273.195, P<0.0001). The probability of ticks being dead was significantly greater for I. scapularis, relative to A. americanum, over the 8-day observation period (x²=138.370, P<0.0001).

A total of 572 out of 840 I. scapularis were recovered (68.1%) of which 371 (64.9%) were female. Of the recovered females, 66% (n=245) were attached; in contrast, 85.6% of males (n=172) were detached. Recovery within test groups totaled 75% (T48), 78.9% (T120) and 50.4% (control group). Recovery was more difficult within the control group, with a discrepancy in the number of males (n=24), relative to the treatment groups (n=81, n=96). A total of 654 of 840 A. americanum were recovered (77.9%) with test groups totaling 78.2% (T48), 71.1% (T120), and 84.3% (control group). The A. americanum ticks had a greater tendency to remain attached, with 88% of females (n=316) and 79.3% of males (n=234) still attached by the end of the 8-day post-attachment period.

### Tick Feeding Status and Condition

Tick feeding status and condition data are explicitly presented in Table 2 and were used to calculate all sums and percentages presented in this section. Within the T48, T120, and control groups, 11.6% (n=15), 1.6% (n=2), and 45.3% (n=53), respectively, of recovered female I. scapularis were alive. All flat I. scapularis females collected at day 8 post-attachment were dead regardless of test group, with a far greater number of flat females collected in the T48 (n=65) and T120 (n=103) groups than in the control group (n=21). Survivorship was significantly greater within the control group, relative to the treatment groups (x²=82.696, P<0.0001). Within the T48 and T120 groups, the greatest proportion of I. scapularis females collected were flat and dead, representing 50.4% (n=65) and 82.4% (n=103) of the respective totals collected. There was a significant difference in the feeding status of the female ticks of the control group relative to those of T48 and T120 groups (x²=114.495, P<0.0001). Live and fully engorged females accounted for the largest proportion of ticks within the control group (38.5%, n=45), a by-product of no exposure to FDF, with dead and partially engorged females accounting for the second largest proportion (35%, n=41). Only two of 201 males collected by day 8 post-attachment were alive (1%). For A. americanum, in the T48, T120 and control groups, 13% (n=16), 5.9% (n=7) and 99.1% (n=116), respectively, of recovered females were alive. Tick survivorship was significantly greater within the control group relative to the treatment groups (x²=265.729, P<0.0001). Within the treatment groups, the proportions of flat and dead and partially engorged and dead females were relatively similar and represented the majority of females collected in the T48 (87%, n=107) and T120 (94.1%, n=112) groups. There was a significant difference in the feeding status of female ticks in the control group relative to female ticks in the T48 and T120 groups (x²=24.967, P<0.0001). Within the control group, 80.3% of the females collected were partially engorged and alive. In total, 57.6% of A. americanum males collected were alive at day 8 post-attachment, with 44.8% (n=43), 17.5% (n=14) and 95% (n=113) of males found alive in the T48, T120 and control groups, respectively.

I. scapularis Oviposition and Larval Hatching

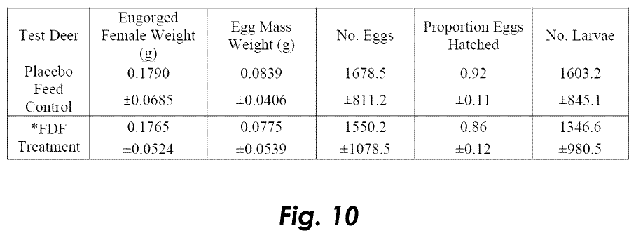

A summary of the average weights of engorged I. scapularis females and egg masses, the approximate number of eggs and the approximate number of emerging larvae is presented in FIG. 10. Although the control group yielded slightly heavier engorged females and egg masses, and larger numbers of eggs and larvae, relative to the treatment groups, it was determined that these differences were not statistically significant.

### Mortality/Efficacy Estimates

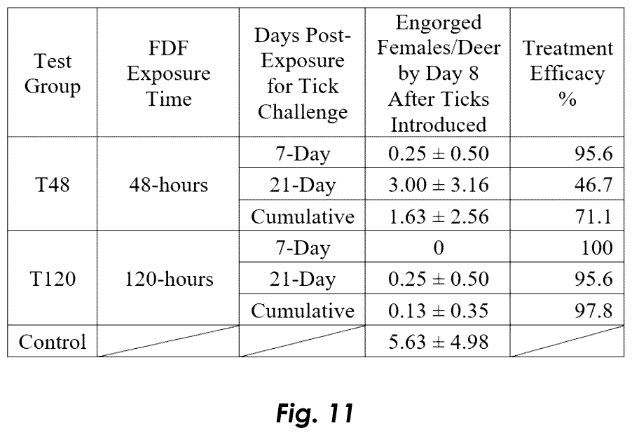

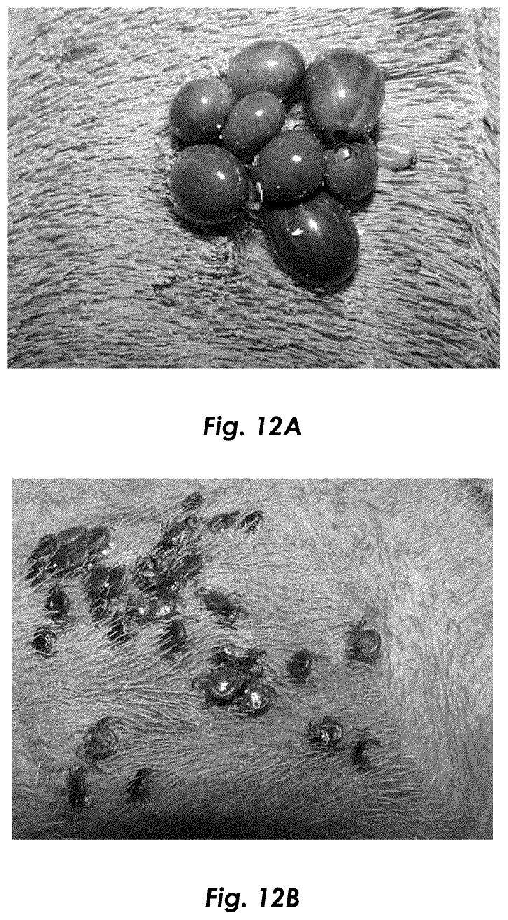

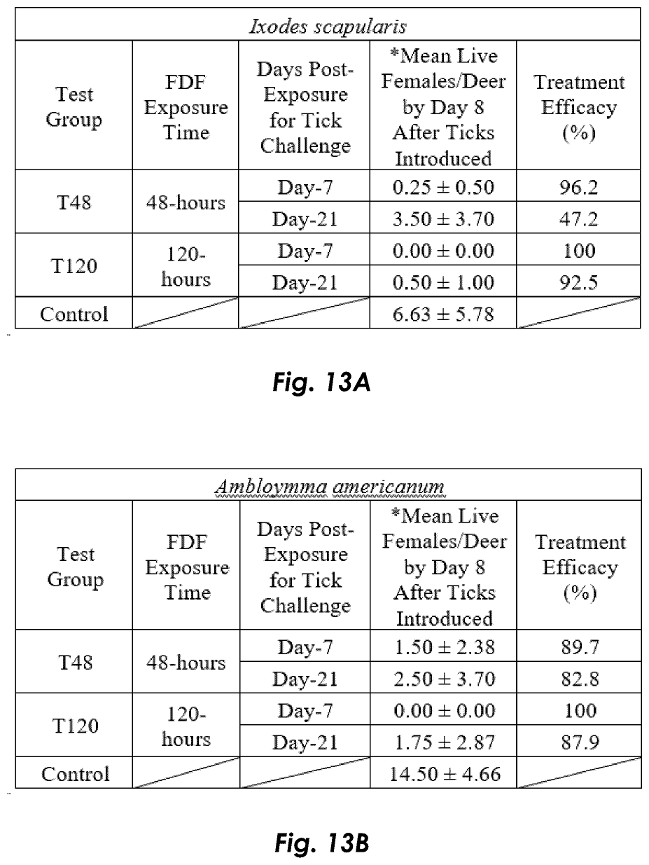

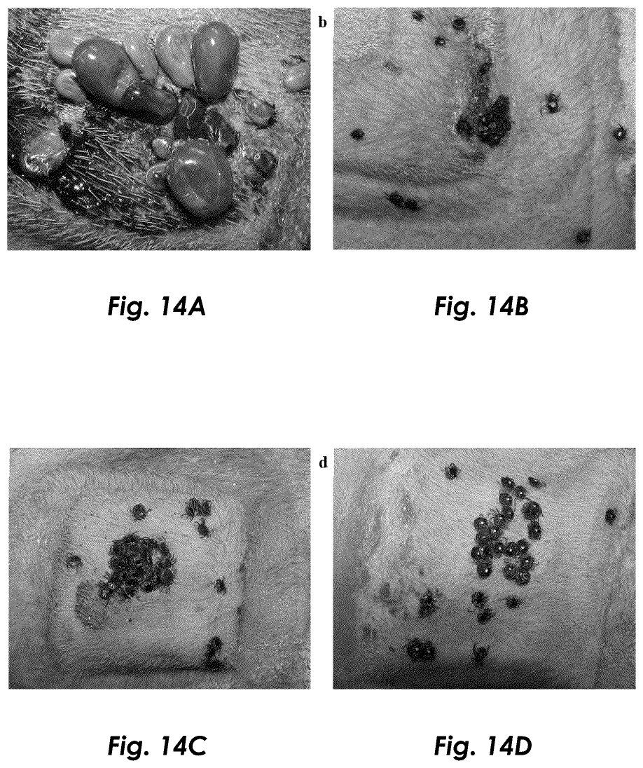

Treating deer with FDF had a significant impact on the mortality/survivorship of both tick species. The average number of engorged I. scapularis females detaching per deer within each test group and subgroup, and resulting efficacy estimates, are presented in FIG. 11. A. americanum blood fed markedly slower than I. scapularis, and thus efficacy in preventing fully engorged females from detaching was not attainable during the 8-day post-attachment period (FIGS. 12A and 12B). The average number of live female I. scapularis and A. americanum (attached and detached) observed per deer per test subgroup and resulting efficacy estimates are presented in FIGS. 13A and 13B. Representative photos of attached I. scapularis and A. americanum within the control and treatment groups are presented in FIGS. 14A through 14D.

FDF Efficacy Against I. scapularis

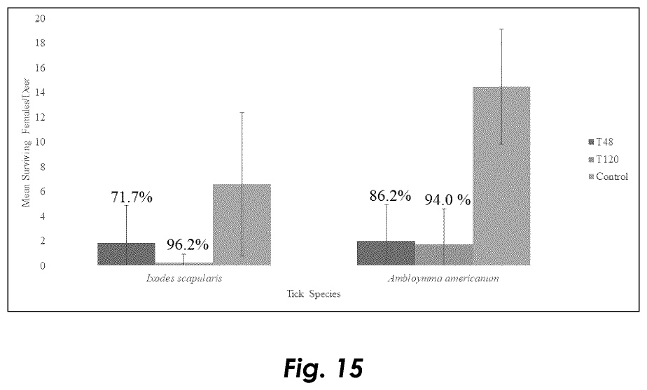

Treating deer with the FDF for 48 h resulted in 95.6% efficacy in preventing ticks placed on deer on day 7 post-FDF exposure from feeding to engorgement and detaching, relative to the control group (FIG. 11). It also led to a 96.2% reduction in survivorship of females, relative to the control group (FIGS. 13A and 13B). Efficacy of 48-h FDF treatment dropped for ticks placed on deer on day 21 post-FDF exposure, with a 46.7% reduction in detaching engorged ticks (FIG. 11) and a 47.2% reduction in survivorship of females (FIGS. 13A and 13B), relative to the control group. Treating deer with FDF for 120 h resulted in 100% efficacy in reducing female survivorship (FIGS. 13A and 13B) and in preventing ticks placed on deer on day 7 post-FDF exposure from feeding to engorgement and detaching (FIG. 11). For ticks placed on deer on day 21 post-FDF exposure, the 120-h FDF treatment efficacy in reducing detached engorged ticks and overall survivorship was 95.6% and 92.5%, respectively. When combining the subgroups for timing of tick placement on deer, the 48-h FDF treatment resulted in a 71.1% reduction in detached engorged females (FIG. 11) and a 71.7% reduction in survivorship (FIG. 15), and the 120-h FDF treatment resulted in a 97.8% reduction in engorgement and detachment (FIG. 11) and a 96.2% reduction in survivorship (FIG. 15).

FDF Efficacy Against A. americanum

Treating deer with FDF for 48 h resulted in an efficacy of 89.7% and 82.8% when ticks were placed on deer on day 7 and day 21 post-FDF exposure, respectively (FIGS. 13A and 13B). Treating deer with FDF for 120 h resulted in an efficacy of 100% and 87.9% when ticks were placed on deer on day 7 and day 21 post-FDF exposure, respectively (FIGS. 13A and 13B). When combining subgroups for timing of tick placement on deer, the 48-h and 120-h FDF treatments resulted in 86.2% and 94% reductions in survivorship, respectively (FIG. 15).

### Fipronil Concentrations in Deer

The majority of pure fipronil was metabolized or excreted. Fipronil sulfone was the metabolite detected above the LOQ.

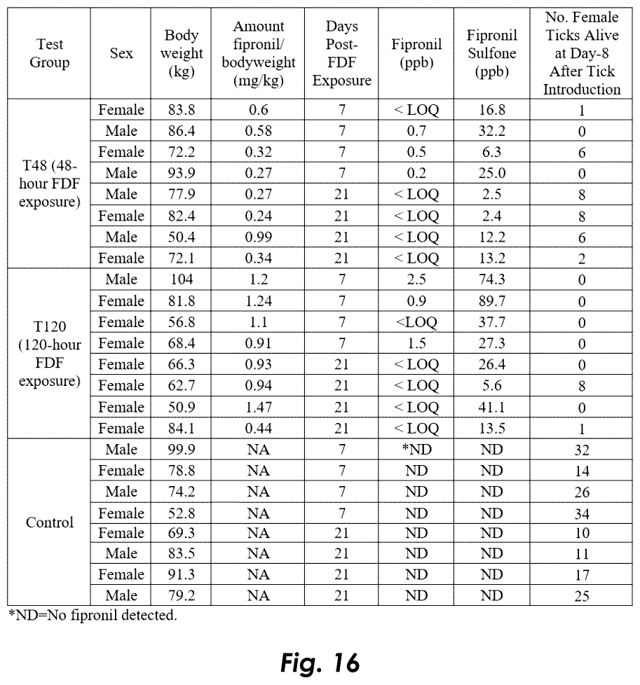

The Cp values for each test deer are presented in FIG. 16. All treated deer had fipronil sulfone detectable >LOQ. The Cp values were highest in the 120-h exposure group (T120), with the average Cp being 57.3 ppb (day 7 post-FDF exposure) and 21.7 ppb (day 21 post-FDF exposure). For the 48-h exposure group (T48), the average Cp values were 20.1 ppb (day 7 post-FDF exposure) and 7.6 ppb (day-21 post-FDF exposure). The reduction from day 7 to day 21 after FDF exposure supports the reduction in efficacy observed. There was a significant linear correlation between Cp and the mg/kg fipronil that was consumed by individual deer (r²=0.6150; P<0.0001). Additionally, there were correlations between Cp and the number of surviving female I. scapularis (r²=0.2057; P<0.0260) and A. americanum (r²=0.3573; P<0.0020) per deer. However, tick survivorship appeared to decrease exponentially rather than linearly in response to elevated Cp, with no female ticks surviving when Cp in plasma was ≥25.0 ppb.

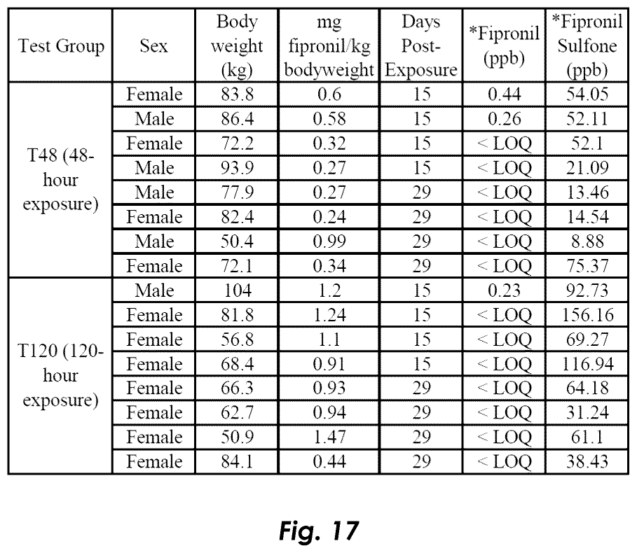

Explicit Cf values for each deer are available in FIG. 17. All treated deer had Cf>LOQ. The Cf values were highest in the 120-h exposure group (T120), with the average Cf being 108.8 ppb (day 7 post-FDF exposure) and 48.7 ppb (day 21 post-FDF exposure). For the 48-h exposure (T48), the average Cf values were 44.8 ppb (day 7 post-FDF exposure) and 28.1 ppb (day 21 post-FDF exposure). Similar to Cp, there was a significant linear correlation between Cf and the mg/kg fipronil that was consumed by individual deer (12=0.5680; P<0.0001).

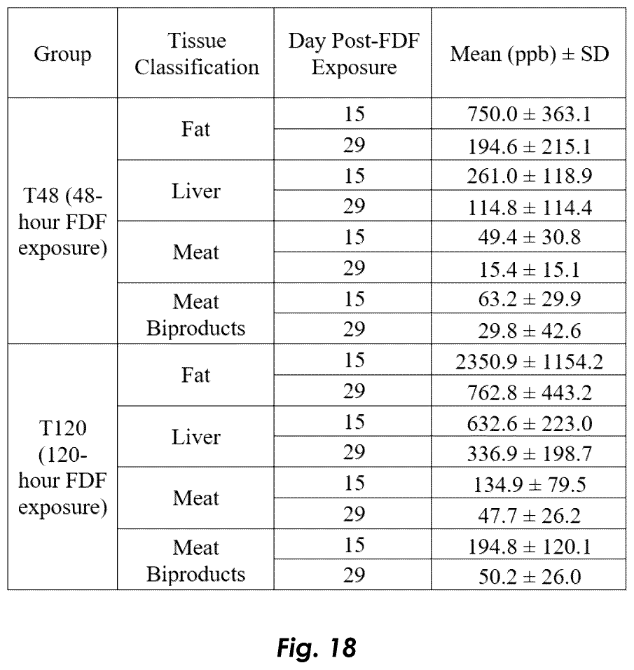

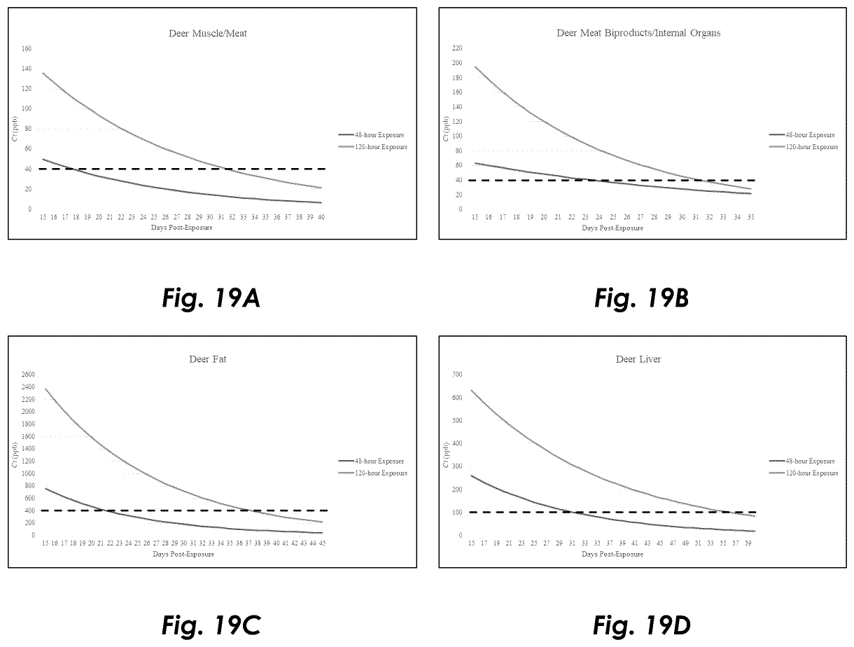

The Ct values were significantly different among various tissue classifications (x²=81.591, df=3, P<0.0001). Fipronil is a lipophilic compound, and the Ct in fat was determined to be significantly higher relative to that in meat/muscle tissues (Z=−7.905, P<0.0001), meat by-products (Z=−5.906, P<0.0001) and liver (Z=−2.516, P=0.0119). Additionally, Ct values in liver tissues were significantly greater than the Ct values in meat (Z=−4.918, P<0.0001) and meat by-products (Z=−3.816, P<0.0006). Deer exposed to fipronil for 120 h had significantly higher Ct in fat (Z=2.848, P=0.0044), meat (Z=5.521, P<0.0001) and meat by-products (Z=2.224, P=0.0261) (liver was not significant), relative to deer exposed to fipronil for 48 h. A summary of the Ct in tissues is given in FIG. 18. Ct was present >LOQ in all tissues collected from deer in the treatment groups. For T48 (48-h exposure), differences in Ct values obtained from tissues collected at day 15 and day 29 were significant (Z=−4.873, P<0.0001), with Ct values at day 29 being 74% (fat), 56% (liver), 68.9% (meat) and 52.8% (meat by-products) less than at day 15. The difference in Ct between day 15 and day 29 was significant (Z=4.287, P<0.0001) in the T120 (120-h exposure) group also, with Ct at day 29 being 67.5% (fat), 46.7% (liver), 64.7% (meat) and 74.2% (meat by-products) less than at day 15. The estimates suggested that the respective deer exposed to FDF for 48 h and 120 h would have post-exposure Ct values degrade to below the EPA MRLs within 22 and 38 days (fat), 32 and 56 days (liver), 18 and 32 days (meat) and 24 and 32 days (meat by-products), respectively (FIGS. 19A through 19D).

### DISCUSSION

White-tailed deer represent a key reproductive host for I. scapularis and A. americanum, and host-targeted approaches such as the application of an oral acaricide have the potential to dramatically reduce tick population abundance and subsequent risk of pathogen-infected tick bites. The results of the study detailed herein suggest that a deer feed containing a nominal concentration of 0.0025% fipronil, presented to white-tailed deer for 48 h and 120 h, can control female I. scapularis and A. americanum ticks parasitizing at day 7 and day 21 post-exposure, with the efficacy dependent upon the amount consumed and the subsequent concentration of fipronil sulfone in the plasma. The fact that no significant differences were detected when comparing FDF and placebo consumption suggested that the inclusion of fipronil in the formulation did not reduce feed palatability. Results suggested that 100% of I. scapularis and A. americanum were eliminated when Cp was present at >25 ppb. Additionally, in total, only one live female tick (A. americanum) was collected from a deer having Cp at 13.5 ppb. The current Cp values are higher than the Cp values determined to control 100% I. scapularis larvae parasitizing P. leucopus (≥8.8 ppb) [Poché, D. M., Dawson, K., Tseveenjav, B. et al. Efficacy of low-dose fipronil bait against blacklegged tick (Ixodes scapularis) larvae feeding on white-footed mice (Peromyscus leucopus) under simulated field conditions. Parasites Vectors 14, 459 (2021)] but are markedly lower than the fluralaner concentrations in plasma reportedly required to control 97% (13000 ppb) and 94% (4000 ppb) I. scapularis larvae parasitizing Peromyscus maniculatus (a species closely phylogenetically related to P. leucopus) [Pelletier, J., Rocheleau, J P., Acnishaenslin, C. et al. Evaluation of fluralaner as an oral acaricide to reduce tick infestation in a wild rodent reservoir of Lyme disease. Parasites Vectors 13, 73 (2020)]. The integration of rodent-targeted and deer-targeted oral acaricide usage may have the potential to reduce tick burden on the primary pathogen host and key reproductive hosts for the I. scapularis vector of B. burgdorferi and, if properly implemented, could potentially have a significant impact on vector abundance and B. burgdorferi infection prevalence in the areas where applied.

The efficacy of FDF in controlling ticks at day 7 post-exposure was undeniable, with 48-h and 120-h exposure successfully controlling both tick species. For I. scapularis, near-complete control was obtained, with only one detached engorged I. scapularis being collected in total (48-h exposure only). The treatment was also effective against A. americanum with complete control obtained in the 120-h exposure group and nearly 90% control within the 48-h exposure group. At day 21 post-exposure, 120-h fipronil exposure resulted in a high efficacy against I. scapularis, with only one detached engorged female being collected in total. The effectiveness of the 48-h exposure and 120-h exposure against A. americanum were relatively similar at day 21 with >80% efficacy for each. A. americanum blood feed for an extended duration, relative to I. scapularis, with approximately 70% of females of the former species needing 12-16 days to reach full engorgement which is why the A. americanum used herein had a greater tendency, relative to I. scapularis, to remain attached throughout the 8-day post-attachment period. Thus, this species was not monitored until full engorgement and detachment. Considering these females may have fed for an additional 4-8 days, it may be suspected that efficacy would have increased and may have reached 100% within all treatment groups. For the purposes of the study described herein, it was determined that both tick species could be evaluated concurrently. However, if explicit engorgement and detachment data are desired for A. americanum in the future, exclusively evaluating this species and extending the post-attachment period by several days may need to be considered. The above values satisfy efficacy requirements previously outlined by the EPA for federal approval which suggest an efficacy of 80-100% against tick vectors [United States Environmental Protection Agency (EPA). OPPTS 810.3300: Treatments to Control Pests of Humans and Pets. Washington, DC: EPA; 1998]. If the product proves to be palatable under field conditions as well, and thus can reach a sizable proportion of deer, the fact that FDF was effective up to day 21 post-exposure indicates that the product could be utilized relatively infrequently under field conditions, which would reduce the amount of acaricide going into the environment, thereby reducing risk of exposure to non-target species and bioaccumulation. From a management perspective, the above results are encouraging.

Direct resistance of host-seeking arthropods to fipronil is considered improbable, and fipronil's effectiveness at low concentrations may allow for reduced application rates which pose reduced risk to non-target organisms, relative to other candidate compounds such as malathion and carbaryl. The acute oral LD₅₀ (lethal dose for 50% of test animals/subjects) of fipronil reported for representative mammal species is 97 mg/kg (rats). Fipronil consumption by deer in the current study ranged from 0.24 mg/kg to 0.99 mg/kg for the 48-h exposure and 0.44 mg/kg to 1.47 mg/kg for the 120-h exposure (FIG. 8). The rate of fipronil exposure may be reduced because of the low concentration of fipronil in FDF (0.0025%). As an example, a deer in the T120 group weighing 104 kg consumed 5 kg (5000 g) of FDF, which amounted to 1.2 mg/kg fipronil consumed (FIG. 8). This same deer would need to consume >403 kg of FDF in one sitting to ingest enough fipronil to exceed the oral LD₅₀ for mammalian species, a feat that would be highly improbable. The ability of FDF to be applied at relatively low frequencies, in combination with the low dose of fipronil in the formulation, may reduce the risk to non-target species, such as raccoons, rodents or birds, should they come into contact with FDF. However, field applications conducted using elevated, species-specific deer feeders may considerably reduce or prohibit access by non-target species, including potentially more sensitive animal species, such as rabbits. Future studies involving field deployment of FDF may carefully consider the application rates and explicitly monitor non-target species within treated areas to ensure reduced environmental risk. Considering that fipronil has proven to be effective against vectors of human disease, such as fleas, mosquitoes and phlebotomine sand flies, a focus of future research may include investigating the impact of fipronil treatment on other blood-feeding arthropods associated with deer, such as mosquito species Ceratopogonidae, and deer keds.

While the efficacy results of the study detailed herein meet federal recommendations, LC/MS analysis of tissues suggest that the concentrations of fipronil sulfone in various tissues may be further investigated and that appropriate modifications to management plans may be considered. The results suggest that fipronil sulfone levels will fall below the EPA-established MRL values within a moderate time frame, with fipronil degrading most slowly in the liver. It does not appear that the US FDA and US EPA have established MRL values for deer. Thus, MRL values for ruminant cattle were considered to be the most appropriate when evaluating the results. Venison liver consumption in general may not be recommended due to possible liver fluke (Fascioloides magna) infection or contamination from heavy metals, such as cadmium. It is noted that the deer in this study were raised in captivity. Factors such as environmental conditions, forage availability and stressors may influence fat stores, digestion and metabolic rates in white-tailed deer, and these factors may differ in wild populations, relative to those raised in captivity. Thus, fipronil metabolism and degradation in wild deer may differ from that in captive deer, and this may be worth investigating in future research. By using these collected data to attempt to predict exponential degradation of fipronil, some initial assumptions to aid in management plans can be made. If the treatment strategies in this pen study are utilized in the field, FDF may work most efficiently in states where Lyme disease is endemic and where there are sizable peaks of adult I. scapularis in the spring, with simultaneous targeting of peaks in A. americanum. Treating in the spring and early to mid-summer would allow adequate time for fipronil withdrawal in deer tissues prior to the fall deer hunting season, which coincides with a peak in fall activity of I. scapularis adults. Two factors make A. americanum an ideal target for FDF treatment: (i) all three life stages of A. americanum take blood meals from white-tailed deer; and (ii) each life stage has peak activity which occurs outside of the fall deer hunting season. Additionally, modifications may be made in specific scenarios, such as blending FDF with untreated placebo or whole corn to dilute the fipronil concentration. A lower fipronil concentration (such as 0.0005%) could allow for FDF to be presented for extended durations under field conditions concurrently with peak adult tick activity or could potentially increase the potential for reduced/short-term treatment during hunting season. Agent-based vector-host associative modelling could be useful in predicting the potential of various FDF treatment scenarios. Ultimately, a field trial may be necessary to confirm the concentrations of fipronil sulfone in the tissues of wild deer and to determine the best course of action for future management plans that might involve the use of FDF.

Possible alternative treatment approaches in regions where concerns regarding chronic wasting disease (CWD) and bovine tuberculosis (bTB) preclude deer baiting may be investigated. The concern may be centered primarily upon unnatural congregation of large groups of deer that can result from baiting, which can subsequently increase the risk of direct contact transmission of CWD and bTB. Similar to vaccine deployment strategies being explored in the Midwest, an alternative approach may be to develop a large grid of evenly placed feeders, each containing a small quantity of FDF (approx. 0.5 kg) to target a few deer, rather than large groups, thus reducing congregation. The logistics of such a strategy may need to be further explored during future field testing.

In past studies, many researchers have explicitly described difficulties related to manual tick feeding on animals. The capsule sizes and dimensions utilized herein were adequate to maintain 20 mating pairs of I. scapularis, as the surface area of the capsules (7×7 cm) and the capsule depth (18 mm) exceeded the surface area (5×5 cm) and capsule depth (8 mm) of similar capsules used for infesting rabbits with 20 I. scapularis mating pairs. While recovering ticks on the deer was relatively successful, there were still issues with damage or loss of I. scapularis, particularly within the control group. Because FDF increased mortality within the treatment groups, deer in the treatment groups presumably experienced reduced irritation with fewer actively engorging females, which made the deer less likely to attempt to dislodge the capsules. Given the small size of I. scapularis males, minor shifting of the capsules within the control group apparently was enough to promote escape. The increased irritation caused by actively engorging females, and subsequent shifting of capsules, caused many females to become damaged/popped, or asphyxiated in the coagulated blood of popped females, which resulted in an increased number of dead, partially engorged females. Thus, the formula described by Abbott [Abbott W S. A method of computing the effectiveness of an insecticide. J Econ Entomol. 1925; 18:265-7] was critical in estimating the efficacy by accounting for tick survivorship in the control group. This was not an issue with A. americanum, given they feed much more slowly. However, while the capsule depth exceeded the specifications recommended by Almazan et al. [Almazán C, Bonnet S, Cote M, Slovák M, Park Y, Šimo L. A versatile model of hard tick infestation on laboratory rabbits. J Vis Exp. 2018; e57994] (10 mm), it is suspected that A. americanum damage may have become a considerable issue if they fed to engorgement considering they are a much larger tick, weighing >600 mg on average at full engorgement. Additional modifications may be made to these capsules in future studies to continue to improve tick attachment, recovery and survivorship. While the issues described limited the ability to determine significant FDF impact on oviposition success and larval emergence, it is worth reiterating that the approximate eggs laid/female and the approximate larvae/female within the treatment groups were slightly reduced, relative to those of the control group, and that this may want to be investigated further in future studies. If a field trial is performed in which FDF is presented to wild deer, removing engorged females from treated deer in the wild and monitoring them for oviposition success may be considered.

### CONCLUSIONS

The results of the present study demonstrate the potential usefulness of a fipronil-based oral acaricide in controlling at least two medically important tick species by providing insights into the potential for control up to 21 days post-exposure. The results on fipronil residue in tissues, plasma and feces also provide much needed insights into the fate of fipronil and its metabolites and provide a potential blueprint for future trials. The next logical step would be a field trial in which wild deer would be exposed to FDF at pre-determined exposure points. Deer would then be sedated in the field and ectoparasites would be collected to determine the impact on parasitizing tick burden. A subsample of deer would also be euthanized, and tissues would be obtained to determine the fate of fipronil in wild deer. If this proof-of-concept field work proves successful, a multi-year large-scale field trial would be the next logical step. Confirming efficacy and safety of this product under field conditions could pave the way for federal approval of a product available for large-scale tick control. A fipronil product targeting wild ruminants on a broad scale has the potential for a variety of beneficial applications. Previous research has indicated the effective use of low-dose oral fipronil baits in controlling ticks, fleas, sand flies and mosquitoes parasitizing a variety of mammalian hosts, including rodents and ruminant cattle. Other tick species of medical or veterinary importance, such as cattle ticks (Rhipicephalus annulatus, R. microplus) and H. longicornis, also utilize white-tailed deer as a blood-meal host and would be susceptible to this treatment as well. Thus, researchers should explore usage of FDF for controlling other arthropod vectors beyond those investigated in the current study. The ultimate goal is to produce a federally approved product for use in controlling arthropods infesting white-tailed deer. If US FDA approval is granted for a new animal drug, FDF could provide a useful means of controlling multiple arthropod species, parasitizing wild ruminants, capable of vectoring disease agents transmissible to humans.

As can be easily understood from the foregoing, the basic concepts of the present invention may be embodied in a variety of ways. The invention involves numerous and varied embodiments of a vector control composition and methods for using the same.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “formulation” should be understood to encompass disclosure of the act of “formulating”—whether explicitly discussed or not—and, conversely, were there effectively disclosure of the act of “formulating,” such a disclosure should be understood to encompass disclosure of a “formulation” and even a “means for formulating.” Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

Moreover, for the purposes of the present invention, the term “a” or “an” entity refers to one or more of that entity unless otherwise limited. As such, the terms “a” or “an”, “one or more” and “at least one” can be used interchangeably herein.

Thus, the applicant(s) should be understood to claim at least: i) each of the vector control compositions and methods for using the same herein disclosed and described, ii) the related methods disclosed and described, iii) similar, equivalent, and even implicit variations of each of these compositions and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice versa as necessary to define the matter for which protection is sought by this application or by any subsequent application or continuation, division, or continuation in part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation in part application thereof or any reissue or extension thereon.

Additionally, the claims set forth in this specification are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. A vector control composition, comprising:
an active agent effective to decrease the fitness of a vector capable of transmitting a pathogen;
wherein said active agent comprises a low dose of an acaricide;
wherein an amount of said low dose of said fipronil is not greater than about 0.005% by weight of said composition; and
wherein said active agent is formulated for oral delivery.

2. The composition of claim 1, wherein said vector comprises an acarine.

3. (canceled)

4. The composition of claim 2, wherein said acarine comprises a tick.

5-14. (canceled)

15. The composition of claim 1, wherein said low dose of said acaricide is delivered to said vector via a host of said vector.

16. The composition of claim 15, wherein said host comprises a mammal.

17-18. (canceled)

19. The composition of claim 1, wherein said acaricide comprises a pyrazole acaricide.

20. The composition of claim 19, wherein said pyrazole acaricide comprises a phenylpyrazole acaricide.

21-22. (canceled)

23. The composition of claim 20, wherein said phenylpyrazole acaricide comprises fipronil.

24. The composition of claim 1, wherein said low dose of said fipronil is effective to decrease said fitness of said vector upon ingestion.

25. The composition of claim 24, wherein said low dose of said fipronil is formulated for oral delivery to said vector.

26. The composition of claim 25, wherein said low dose of said fipronil is formulated for ingestion by said vector during blood-feeding on a host.

27. The composition of claim 26, wherein said low dose of said fipronil is effective to decrease said fitness of said vector upon ingestion of said composition by said host.

28. The composition of claim 27, wherein said composition is formulated for oral delivery to said host.

29-47. (canceled)

48. The composition of claim 15, wherein said composition is formulated as a comestible composition for ingestion by said host.

49. The composition of claim 48, wherein said composition is formulated as feed for said host.

50. The composition of claim 49, further comprising a carrier palatable to a target said host and edible by said target host.

51-53. (canceled)

54. The composition of claim 49, wherein said feed comprises a substantially solid form.

55. (canceled)

56. A vector control composition, comprising:
an active agent effective to decrease the fitness of a vector capable of transmitting a pathogen;
wherein said active agent comprises an acaricide; and
wherein said active agent is formulated for oral delivery.

57. The composition of claim 56, wherein said active agent consists of said acaricide.

58. (canceled)

59. The composition of claim 56, wherein said active agent consists of a low dose of said acaricide.

60-77. (canceled)

78. A method of decreasing the fitness of a vector capable of transmitting a pathogen, comprising:
delivering to a host of said vector an effective amount of a vector control composition comprising:
an active agent effective to decrease said fitness of said vector;
wherein said active agent comprises a low dose of an acaricide; and
wherein said active agent is formulated for oral delivery;
wherein said method decreases said fitness of said vector relative to an untreated vector.

79-83. (canceled)
